(19) United States
(12) Patent Application Publication
(10) Pub. No.: US 2014/0336428 A1
(43) Pub. Date: Nov. 13, 2014

(54) RECYCLE GAS SCRUBBING USING IONIC LIQUIDS

(71) Applicant: UOP LLC, Des Plaines, IL (US)

(72) Inventors: Soumendra M. Banerjee, New Delhi (IN); Alakananda Bhattacharyya, Glen Ellyn, IL (US); Erin M. Broderick, Evanston, IL (US)

(73) Assignee: UOP LLC, Des Plaines, IL (US)

(21) Appl. No.: 13/892,014

(22) Filed: May 10, 2013

(51) Int. Cl. B01D 53/14; C07C 7/11

(52) U.S. Cl. CPC B01D 53/1475; B01D 53/1425; C07C 7/11
USPC 585/251; 95/177; 585/310

(57) ABSTRACT

A method of removing CO₂ from a gaseous mixture using ionic liquids is described. The ionic liquids can be regenerated by pressure reductions. A method of processing a renewable feedstock using the ionic liquids to remove CO₂ is also described.

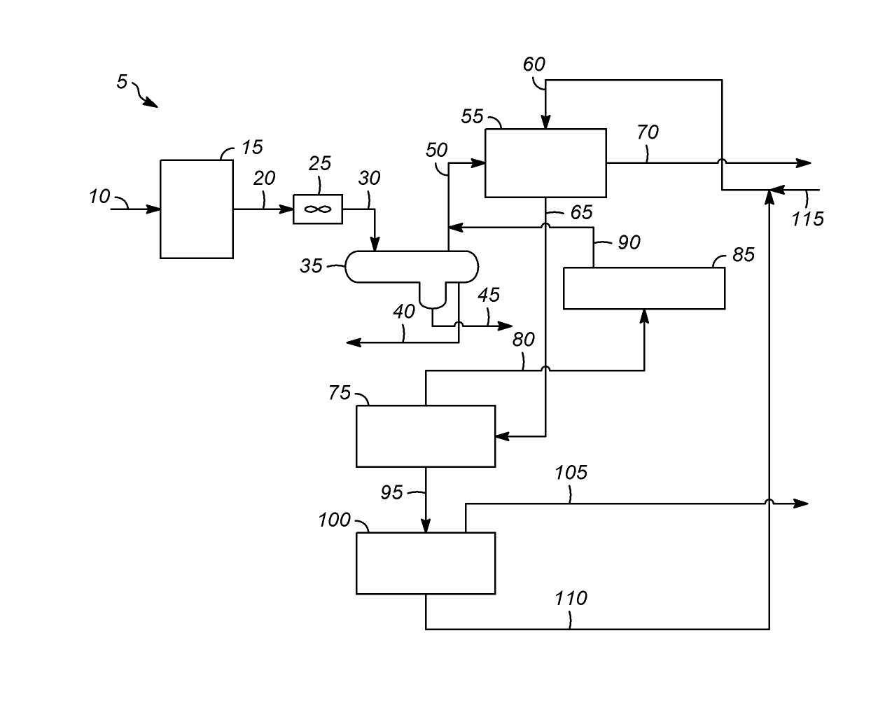

### BACKGROUND OF THE INVENTION

The search for alternative, sustainable sources of energy for the transportation sector has been spurred by the concern over limited fossil fuel resources and the effect of CO₂ emissions on global warming. The world-wide development of alternative fuel sources is also driven by rising petroleum prices as well as government regulations and incentives.

One such source is what has been termed biorenewable sources, which include plant oils such as corn, rapeseed, canola, and soybean oils, and greases such as inedible tallow, yellow and brown greases. The common feature of these sources is that they are composed of triglycerides and Free Fatty Acids (FFA). Both of these compounds contain n-paraffin chains having 10 to 20 carbon atoms. The n-paraffin chains in the tri-glycerides or FFAs can also be mono-, di- or poly-unsaturated.

Despite the growth in renewable fuels, there has been limited integration of renewable fuels into petroleum refineries. Most refineries for renewable fuels are set up as stand alone units for the processing of neat vegetable oils. For example, the hydrotreating process for treating neat vegetable oil available from UOP LLC produces high cetane diesel that can be blended effectively into the refiner's diesel.

In contrast to conventional petroleum hydrotreating processes, hydrotreating renewable feeds produces high levels of methane, propane, water, carbon monoxide, and carbon dioxide. These gases must be removed by chemical transformation, by a gas cleaning step, such as an amine wash, or by increasing the purge gas rate. If the gases are not properly removed, they will result in a decreased hydrogen partial pressure, resulting in reduced catalyst activity. The CO, which cannot be removed by an amine wash unit, will build up in the treat gas, requiring a high purge rate or another means of treat gas purification. Liquid water and CO₂ can react in the reactor effluent train to form carbonic acid, which must be handled properly to avoid increased corrosion rates.

While CO can be handled by proper selection of the catalyst or by methanation, it is necessary to remove the CO₂ from the recycle gas. Commercial amine solutions used for this purpose monoethanolamine (MEA), diethanolamine (DEA), and N-methyldiethanolamine (MDEA). In this process, the amine solution circulates in a loop between the adsorption of the CO₂ and the regeneration of the amine solvent.

When such a unit is added to a petroleum refinery, the presence of the CO₂ presents a challenge because the existing amine regeneration unit may not be designed to handle the CO₂. Thus, two separate amine regeneration systems would be required. In addition, there could be an increased energy requirement for the regenerator reboiler because additional energy could be required to break the CO₂-rich amine chemical bond. Depending on the amine involved and the circulation rate of the lean amine, this increase could be substantial. Furthermore, some amines react with CO₂ in ways that are not reversible in the regeneration step.

Therefore, there is a need for a CO₂ removal process.

### SUMMARY OF THE INVENTION

One aspect of the invention is a method for removing CO₂ from a gaseous mixture comprising CO₂, hydrocarbon vapor, and hydrogen. In one embodiment, the method includes contacting the gaseous mixture with an absorbing medium in an absorption zone at a first pressure in range of about 2 MPa (g) to about 10 MPa (g) so that the CO₂ is absorbed by the absorbing medium forming a stream of absorbing medium rich in CO₂ and the hydrocarbon vapor, the stream of absorbing medium rich in CO₂ and the hydrocarbon vapor at a first pressure, the absorbing medium comprising an ionic liquid wherein the ionic liquid is not a pyrrolidinone-based ionic liquid. The pressure of the stream of absorbing medium rich in CO₂ and the hydrocarbon vapor is reduced from the first pressure to a second pressure lower than the first pressure to release the hydrocarbon vapor from the absorbing medium rich in CO₂ and the hydrocarbon vapor to form a stream of absorbing medium rich in CO₂. The pressure of the stream of absorbing medium rich in CO₂ is reduced from the second pressure to a third pressure lower than the second pressure to release the CO₂ from the absorbing medium to form a stream of absorbing medium substantially free of hydrocarbon vapor and CO₂.

Another aspect of the invention is a method for processing a renewable feedstock. In one embodiment, the method includes hydrotreating the renewable feedstock in a hydrotreating zone to produce a reactor effluent comprising hydrocarbons, hydrogen, water, CO and CO₂. The reactor effluent is separated in a separation zone at a first pressure in a range of about 2 MPa (g) to about 10 MPa (g) into a liquid stream comprising liquid hydrocarbon and a gaseous stream comprising hydrocarbon vapor, hydrogen, water, CO and CO₂. The gaseous mixture is contacted with an absorbing medium in an absorption zone so that the CO₂ is absorbed by the absorbing medium forming a stream of absorbing medium rich in CO₂ and the hydrocarbon vapor, the stream of absorbing medium rich in CO₂ and the hydrocarbon vapor at the first pressure, the absorbing medium comprising an ionic liquid wherein the ionic liquid is not a pyrrolidinone-based ionic liquid. The pressure of the stream of absorbing medium rich in CO₂ and the hydrocarbon vapor is reduced from the first pressure to a second pressure lower than the first pressure to release the hydrocarbon vapor from the absorbing medium rich in CO₂ and the hydrocarbon vapor to form a stream of absorbing medium rich in CO₂. The pressure of the stream of absorbing medium rich in CO₂ is reduced from the second pressure to a third pressure lower than the second pressure to release the CO₂ from the absorbing medium to form a stream of absorbing medium substantially free of hydrocarbon vapor and CO₂.

### BRIEF DESCRIPTION OF THE DRAWING

The FIGURE illustrates one embodiment of the process of the present invention.

### DETAILED DESCRIPTION OF THE INVENTION

A process has been developed to remove CO₂ from recycle gas using an absorbing medium containing an ionic liquid (IL). The recycle gas mixture is contacted with the ionic liquid, and the ionic liquid absorbs the CO₂ from the mixture. The ionic liquid can be regenerated using multiple flashing steps at progressively lower pressures.

The ionic liquid absorption and regeneration system is self-contained, and it is not mixed with systems for removing other contaminants, such as an amine unit for removing sulfur. It can be easily retrofitted into an existing hydrotreating process for vegetable oil, as discussed below.

The gas mixture is contacted at a first pressure. The pressure is then reduced to a second pressure lower than the first pressure to release the hydrocarbon vapor from the ionic liquid. The pressure is then reduced to a third pressure that is less than the second pressure to release the CO₂ from the ionic liquid. Any pressure lower than the first pressure and suitable to release the hydrocarbon vapor from the ionic liquid can be used for the second pressure. Any pressure lower that the second pressure and suitable to release the CO₂ from the ionic liquid can be used for the third pressure. At the third pressure, the absorbing medium is substantially free of hydrocarbon vapor and CO₂. By substantially free, we mean that there is less than about 5% hydrocarbon vapor and CO₂.

Typically, the first pressure in the range of about 2 MPa (g) to about 10 MPa (g), or about 2 MPa (g) to about 8 MPa (g), or about 2 MPa (g) to about 6 MPa (g), or about 3 MPa (g) to about 10 MPa (g), or about 4 MPa (g) to about 10 MPa (g), or about 4 MPa (g) to about 8 MPa (g). The second pressure is less than the first pressure, so the pressure will depend on what the first pressure was. The second pressure is typically in the range of about 0.5 MPa (g) to about 4 MPa (g), or about 0.5 MPa (g) to about 3 MPa (g), or about 0.5 MPa (g) to about 2 MPa (g), or about 0.5 MPa (g) to about 1 MPa (g), or about 1 MPa (g) to about 2 MPa (g). The third pressure is less than the second pressure, so the pressure will depend on what the second pressure was. The third pressure is typically in the range of 0 MPa (g) to about 2 MPa (g), or 0 MPa (g) to about 1.5 MPa (g), or 0 MPa to about 1 MPa (g), or 0 MPa (g) to about 0.5 MPa (g).

In one embodiment, the first pressure is in a range of about 2 MPa to about 6 MPa, the second pressure is in a range of about 0.5 MPa to about 2 MPa, and the third pressure is in a range of 0 to about 0.5 MPa.

The ionic liquid regeneration does not require the high temperatures needed for the amine absorption and regeneration process (e.g., about 130° C. to about 140° C.).

The FIGURE illustrates one embodiment of the process 5. The renewable feedstock 10 is introduced into the hydrotreating zone 15 which is described below. The effluent 20 from the hydrotreating zone 15 is typically at a temperature in the range of about 120° C. to about 140° C. The effluent 20 is at least partially condensed in condenser 25. The partially condensed effluent 30, which typically is at a temperature in the range of about 40° C. to about 65° C., is sent to separator 35. The separator 35 typically operates at about the same pressure as the hydrotreating zone 15, e.g., about 4 to about 8 MPa (g). The partially condensed effluent 30 is separated into a liquid hydrocarbon stream 40, a water stream 45, and a gas stream 50. The liquid hydrocarbon stream 40 can be sent to a fractionation zone (not shown) to be separated into one or more hydrocarbon fractions, such as a diesel fraction and a naphtha fraction, for use as fuel. The water stream 45 can be sent for further processing (not shown) as needed.

The gas stream 50, which contains hydrocarbon vapors (including methane, and propane), water, CO, and CO₂, is sent to an absorption zone 55 where it is contacted with absorbing medium 60. The absorption zone 55 operates at about the same pressure as the separator 35. The temperature in the absorption zone is typically in the range of about 40° C. to about 65° C.

The gas stream 50 and the absorbing medium 60 can be mixed in the absorption zone 55. The absorption zone 55 typically uses a trayed column for absorption. Alternatively, the gas stream 50 and at least a portion of the absorbing medium 60 (e.g., about 10 to about 15%) can be mixed before entering the absorption zone 55.

Absorbing medium 60 comprises one or more ionic liquids. The absorbing medium optionally includes one or more solvents. Suitable solvents include, but are not limited to, methanol, dimethyl ethers of polyethylene glycol, and N-methylpyrrolidone. The solvent is typically present in an amount of about 1% to about 90%, or about 1% to about 75%, or about 1% to about 50%, or about 1% to about 25%. The addition of a solvent, such as, but not limited to, methanol, dimethyl ethers of polyethylene glycol, or N-methylpyrrolidone, to an ionic liquid decreases the viscosity of the ionic liquid. In certain cases, solvents may also enable regeneration of the ionic liquid and solvent mixture without heat.

The ionic liquid is not a pyrrolidinone-based ionic liquid. The ionic liquid comprises a cation and an anion. The anion complexes with the CO₂ in the recycle gas and effectively removes it. Suitable anions include, but are not limited to, carboxylates, acetates, tosylates, cyanates, halides, sulfates, hydrogen sulfates, sulfonates, sulfonyl imides, phosphates, borates, carbonates, and heterocyclic anions. Suitable cations include, but are not limited to, imidazolium, phosphonium, alcamine, guanidinium, tetraalkylammonium, pyrazolium, pyridinium, sulfonium, piperidinium. In some, embodiments, the ionic liquid can be mixed with amino reagents, for example IL-2-aminoethanol. Adding amines to ionic liquids is described, for example, in US 2012/0063978, which is incorporated herein by reference. In some embodiments, imidazolium-based ionic liquids are not used.

The absorbing medium absorbs the hydrocarbons and CO₂ in the gas mixture forming a stream 65 rich in hydrocarbons and CO₂. Recycle gas stream 70, which contains hydrogen, can be sent to a recycle gas compressor (not shown).

The pressure of the stream 65 rich in hydrocarbons and CO₂ is reduced in a first pressure reduction zone 75 to release the hydrocarbon vapor 80 from the absorbing medium while leaving the CO₂. The pressure is reduced to about 2 MPa in the first pressure reduction zone 75. The hydrocarbon vapor 80 can be compressed in compressor 85, and the compressed hydrocarbon vapor 90 can be mixed with the gas stream 50 and recycled to the absorption zone 55 so that hydrogen and/or hydrocarbons do not escape with the CO₂ in the final pressure reduction.

Stream 95 from the first pressure reduction zone 75 comprises absorbing medium rich in CO₂. Stream 95 is sent to a second pressure reduction zone 100 where the CO₂ is released from the absorbing medium. The pressure is reduced to about atmospheric pressure in the second pressure reduction zone.

The CO₂ rich gas stream 105 can be sent for further processing or use in other areas of the refinery (not shown). The recycled absorbing medium 110 can be combined with make-up absorbing medium 115 (if needed) to form absorbing medium 60. The recycled absorbing medium 110 will typically be in the range of about 40° C. to about 80° C.

The ionic liquid is regenerated by pressure letdown, with no heating needed. However, the stream can be heated, if desired (e.g., the temperature after heating would typically be less than about 100° C., or less than about 90° C., or less than about 80° C.). In contrast, regeneration of amines requires heating with stream to a temperature in the range of about 130° C. to about 140° C.

Either one or both of the recycled absorbing medium 110 or the absorbing medium 60 can be cooled, and compressed before being introduced into the absorption zone 55.

Suitable pressure reduction zones include, but are not limited to, pressure flash drums.

Although the process is illustrated using two pressure reduction zones, additional pressure reduction zones can be used, if desired.

The hydrotreating process relates to a process for producing a hydrocarbon stream useful as fuels, such as diesel fuel and aviation fuel, from biorenewable feedstocks. U.S. Pat. Nos. 7,999,143, 8,003,834, 8,198,492, and 8,329,967 and US Publication No. 2010/0245553, each of which is incorporated herein in its entirely, describe various hydrotreating processes for biorenewable feedstocks. Biorenewable feedstocks include feedstocks other than those obtained from petroleum crude oil. Another term that has been used to describe this class of feedstock is biorenewable fats and oils. The renewable feedstocks that can be used in the present invention include any of those which comprise glycerides and free fatty acids (FFA). Most of the glycerides will be triglycerides, but monoglycerides and diglycerides may be present and processed as well. Another class of compounds appropriate for these processes fatty acid alkyl esters (FAAE), such as fatty acid methyl ester (FAME) or fatty acid ethyl ester (FAEE). Examples of these renewable feedstocks include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, palm oil, mustard oil, cottonseed oil, camelina oil, jatropha oil, crambe oil, tallow, yellow and brown greases, lard, train oil, fats in milk, fish oil, algal oil, sewage sludge, and the like. The glycerides and FFAs of the typical vegetable or animal fat contain aliphatic hydrocarbon chains in their structure which have about 8 to about 24 carbon atoms with a majority of the fats and oils containing high concentrations of fatty acids with 16 and 18 carbon atoms.

The renewable feedstocks may contain a variety of impurities. For example, tall oil is a byproduct of the wood processing industry and tall oil contains esters and rosin acids in addition to FFAs. Rosin acids are cyclic carboxylic acids. The renewable feedstocks may also contain contaminants such as alkali metals, e.g. sodium and potassium, phosphorous as well as solids, water and detergents.

Although the renewable feedstock can be processed, i.e., hydrodeoxygenated, to hydrocarbons without any prior treatments, it can be pretreated the feedstock in order to remove contaminants. Suitable pretreatments include, but are not limited to, contacting the biorenewable feedstock with an ion-exchange resin, mild acid wash, the use of guard beds, filtration, and solvent extraction.

The renewable feedstock is flowed to a first reaction zone comprising one or more catalyst beds in one or more reactors. The term “feedstock” is meant to include feedstocks that have not been treated to remove contaminants as well as those feedstocks purified in a pretreatment zone. In the reaction first zone, the feedstock is contacted with a hydrogenation or hydrotreating catalyst in the presence of hydrogen at hydrogenation conditions to hydrogenate the reactive components such as olefinic or unsaturated portions of the n-paraffinic chains. Hydrogenation and hydrotreating catalysts are any of those well known in the art such as nickel or nickel/molybdenum dispersed on a high surface area support. Other hydrogenation catalysts include one or more noble metal catalytic elements dispersed on a high surface area support. Non-limiting examples of noble metals include Pt and/or Pd dispersed on gamma-alumina or activated carbon. Hydrogenation conditions include a temperature of about 40° C. to about 400° C. and a typical pressure of about 2 MPa absolute to about 13.8 MPa absolute. In another embodiment the hydrogenation conditions include a temperature of about 200° C. to about 300° C. and a pressure of about 2 MPa absolute to about 4.8 MPa absolute. Other operating conditions for the hydrogenation zone are well known in the art.

The catalysts enumerated above are also capable of catalyzing decarboxylation, decarbonylation and/or hydrodeoxygenation of the feedstock to remove oxygen. Decarboxylation, decarbonylation, and hydrodeoxygenation are herein collectively referred to as deoxygenation reactions. Decarboxylation conditions include a typical pressure of about 2 MPa to about 6.9 MPa, a temperature of about 200° C. to about 400° C. and a liquid hourly space velocity of about 0.5 to about 10 hr⁻¹. In another embodiment the decarboxylation conditions include the same relatively low pressure of about 2 MPa to about 6.9 MPa, a temperature of about 288° C. to about 345° C. and a liquid hourly space velocity of about 1 to about 4 hr⁻¹. Since hydrogenation is an exothermic reaction, as the feedstock flows through the catalyst bed, the temperature increases, and decarboxylation and hydrodeoxygenation will begin to occur. Thus, it is envisioned and is within the scope of this invention that all the reactions occur simultaneously in one reactor or in one bed. Alternatively, the conditions can be controlled such that hydrogenation primarily occurs in one bed and decarboxylation and/or hydrodeoxygenation occurs in a second bed. Of course if only one bed is used, then hydrogenation occurs primarily at the front of the bed, while decarboxylation/hydrodeoxygenation occurs mainly in the middle and bottom of the bed. Finally, desired hydrogenation can be carried out in one reactor, while decarboxylation, decarbonylation, and/or hydrodeoxygenation can be carried out in a separate reactor. However, the order of the deoxygenation reactions is not critical.

The reaction product from the hydrogenation and deoxygenation reactions will comprise both a liquid portion and a gaseous portion. The liquid portion comprises a hydrocarbon fraction comprising n-paraffins and having a large concentration of paraffins in the 15 to 18 carbon number range. Different feedstocks will result in different distributions of paraffins. A portion of this hydrocarbon fraction, after separation from the gaseous portion, may be used as the hydrocarbon recycle described above. Although this hydrocarbon fraction is useful as a diesel fuel or diesel fuel blending component, additional fuels, such as aviation fuels or aviation fuel blending components which typically have a concentration of paraffins in the range of about 9 to about 15 carbon atoms, may be produced with additional processing. Also, because the hydrocarbon fraction comprises essentially all n-paraffins, it will have poor cold flow properties. Many diesel and aviation fuels and blending components must have better cold flow properties and so the reaction product is further reacted under isomerization conditions to isomerize at least a portion of the n-paraffins to branched paraffins.

The gaseous portion comprises hydrogen, carbon dioxide, carbon monoxide, water vapor, propane, and perhaps sulfur components such as hydrogen sulfide, nitrogen components such as ammonia, or phosphorous components such as phosphine. The effluent from the deoxygenation zone is conducted to a hot high pressure hydrogen stripper. One purpose of the hot high pressure hydrogen stripper is to selectively separate at least a portion of the gaseous portion of the effluent from the liquid portion of the effluent. As hydrogen is an expensive resource, to conserve costs, the separated hydrogen is recycled to the first reaction zone containing the deoxygenation reactor. Also, failure to remove the water, carbon monoxide, and carbon dioxide from the effluent may result in poor catalyst performance in the isomerization zone. Water, carbon monoxide, carbon dioxide, any ammonia or hydrogen sulfide are selectively stripped in the hot high pressure hydrogen stripper using hydrogen. The hydrogen used for the stripping may be dry, and free of carbon oxides. The temperature may be controlled in a limited range to achieve the desired separation and the pressure may be maintained at approximately the same pressure as the two reaction zones to minimize both investment and operating costs. The hot high pressure hydrogen stripper may be operated at conditions ranging from a typical pressure of about 2 MPa absolute to about 13.8 MPa absolute, and a temperature of about 40° C. to about 350° C. In another embodiment the hot high pressure hydrogen stripper may be operated at conditions ranging from a pressure of about 2 MPa absolute to about 4.8 MPa absolute, or about 2.4 MPa absolute to about 4.9 MPa absolute, and a temperature of about 50° C. to about 350° C. The hot high pressure hydrogen stripper may be operated at essentially the same pressure as the reaction zone. By “essentially”, it is meant that the operating pressure of the hot high pressure hydrogen stripper is within about 1 MPa absolute of the operating pressure of the reaction zone. For example, in one embodiment the hot high pressure hydrogen stripper separation zone is no more than 1 MPa absolute less than that of the reaction zone.

The effluent enters the hot high pressure stripper and at least a portion of the gaseous components, are carried with the hydrogen stripping gas and separated into an overhead stream. The remainder of the deoxygenation zone effluent stream is removed as hot high pressure hydrogen stripper bottoms and contains the liquid hydrocarbon fraction having components such as normal hydrocarbons having from about 8 to 24 carbon atoms. A portion of this liquid hydrocarbon fraction in hot high pressure hydrogen stripper bottoms may be used as the hydrocarbon recycle described below.

A portion of the lighter hydrocarbons generated in the deoxygenation zone may be also carried with the hydrogen in the hot high pressure hydrogen stripper and removed in the overhead stream. Any hydrocarbons removed in the overhead stream will effectively bypass the isomerization zone, discussed below. A large portion of the hydrocarbons bypassing the isomerization zone will be normal hydrocarbons which, due to bypassing the isomerization stage, will not be isomerized to branched hydrocarbons. At least a portion of these normal hydrocarbons ultimately end up in the diesel range product or the aviation range product, and depending upon the specifications required for the products, the normal hydrocarbons may have an undesired effect on the diesel range product and the aviation range product. For example, in applications where the diesel range product is required to meet specific cloud point specifications, or where the aviation range product is required to meet specific freeze point specifications, the normal hydrocarbons from the hot high pressure hydrogen stripper overhead may interfere with meeting the required specification. Therefore, in some applications, it is advantageous to take steps to prevent normal hydrocarbons from being removed in the hot high pressure hydrogen stripper overhead and bypassing the isomerization zone. For example, one or more, or a mixture of additional rectification agents may be optionally introduced into the hot high pressure hydrogen stripper to reduce the amount of hydrocarbons in the hot high pressure hydrogen stripper overhead stream. Suitable example of additional rectification agents include the diesel boiling point range product, the aviation boiling point range product, the naphtha boiling range product, the mixture of naphtha and LPG, or any combinations thereof. These streams may be recycled and introduced to the hot high pressure hydrogen stripper, at a location of the stripper that is above the deoxygenation zone effluent introduction location and in the rectification zone. The rectification zone, if present, may contain vapor liquid contacting devices such as trays or packing to increase the efficiency of the rectification. The optional rectification agent would operate to force an increased amount of the hydrocarbon product from the deoxygenation zone to travel downward in the hot high pressure hydrogen stripper and be removed in the hot high pressure hydrogen stripper bottoms stream instead of being carried with the stripping hydrogen gas into the hot high pressure hydrogen stripper overhead. Other rectification agents from independent sources may be used instead of, or in combination with, the diesel boiling point range product, the naphtha product, and the naphtha and LPG stream.

Hydrogen is a reactant in at least some of the reactions above, and a sufficient quantity of hydrogen must be in solution to most effectively take part in the catalytic reaction. Past processes have operated at high pressures in order to achieve a desired amount of hydrogen in solution and readily available for reaction. However, higher pressure operations are more costly to build and to operate as compared to their lower pressure counterparts. The operating pressure may be in the range of about 2 MPa absolute to about 4.8 MPa absolute which is lower than that found in other previous operations. In another embodiment, the operating pressure is in the range of about 2.4 MPa absolute to about 4.5 MPa absolute, and in yet another embodiment operating pressure is in the range of about 2.7 MPa absolute to about 4.1 MPa absolute. Furthermore, the rate of reaction is increased resulting in a greater amount of throughput of material through the reactor in a given period of time.

In one embodiment, the desired amount of hydrogen is kept in solution at lower pressures by employing a large recycle of hydrocarbon to the deoxygenation reaction zone. Other processes have employed hydrocarbon recycle in order to control the temperature in the reaction zones since the reactions are exothermic reactions. However, the range of recycle to feedstock ratios used herein is determined not on temperature control requirements, but instead, based upon hydrogen solubility requirements. Hydrogen has a greater solubility in the hydrocarbon product than it does in the feedstock. By utilizing a large hydrocarbon recycle the solubility of hydrogen in the combined liquid phase in the reaction zone is greatly increased and higher pressures are not needed to increase the amount of hydrogen in solution. In one embodiment of the invention, the volume ratio of hydrocarbon recycle to feedstock is from about 1:1 or about 2:1 to about 8:1. In another embodiment the ratio is in the range of about 3:1 to about 6:1 and in yet another embodiment the ratio is in the range of about 4:1 to about 5:1.

Although the hydrocarbon fraction separated in the hot high pressure hydrogen stripper is useful as a diesel fuel or diesel fuel blending component because it comprises essentially n-paraffins, it will have poor cold flow properties. Also, depending upon the feedstock, the amount of hydrocarbons suitable for aviation fuel or aviation fuel blending component may be small. Therefore, the hydrocarbon fraction can be contacted with an isomerization catalyst under isomerization conditions to at least partially isomerize the n-paraffins to branched paraffins and improve the cold flow properties of the liquid hydrocarbon fraction. The isomerization catalysts and operating conditions are selected so that the isomerization catalyst also catalyzes selective hydrocracking of the paraffins. The selective hydrocracking creates hydrocarbons in the aviation boiling point range. The effluent of the second reaction zone, the isomerization and selective hydrocracking zone, is a branched-paraffin-enriched stream. By the term “enriched” it is meant that the effluent stream has a greater concentration of branched paraffins than the stream entering the isomerization zone, and preferably comprises greater than 50 mass-% branched paraffins. It is envisioned that the isomerization zone effluent may contains 70, 80, or 90 mass-% branched paraffins. Isomerization and selective hydrocracking can be carried out in a separate bed of the same reactor, described above or the isomerization and selective hydrocracking can be carried out in a separate reactor. For ease of description, the following will address the embodiment where a second reactor is employed for the isomerization and selective hydrocracking reactions. The hydrogen stripped product of the deoxygenation reaction zone is contacted with an isomerization and selective hydrocracking catalyst in the presence of hydrogen at isomerization and selective hydrocracking conditions to isomerize at least a portion of the normal paraffins to branched paraffins. Due to the presence of hydrogen, the reactions may be called hydroisomerization and hydrocracking.

The isomerization and selective hydrocracking of the paraffinic product can be accomplished in any manner known in the art or by using any suitable catalyst known in the art. One or more beds of catalyst may be used. It is preferred that the isomerization be operated in a co-current mode of operation. Fixed bed, trickle bed down flow or fixed bed liquid filled up-flow modes are both suitable. See also, for example, US 2004/0230085 A1 which is incorporated by reference in its entirety. Catalysts having an acid function and mild hydrogenation function are favorable for catalyzing both the isomerization reaction and the selective hydrocracking reaction. Suitable catalysts comprise a metal of Group VIII (IUPAC 8-10) of the Periodic Table and a support material. Suitable Group VIII metals include platinum and palladium, each of which may be used alone or in combination. The support material may be amorphous or crystalline or a combination of the two. The isomerization and selective hydrocracking catalyst may also comprise a modifier selected from the group consisting of lanthanum, cerium, praseodymium, neodymium, phosphorus, samarium, gadolinium, terbium, and mixtures thereof, as described in U.S. Pat. No. 5,716,897 and U.S. Pat. No. 5,851,949. The teachings of U.S. Pat. No. 4,310,440; U.S. Pat. No. 4,440,871; U.S. Pat. No. 4,793,984; U.S. Pat. No. 4,758,419; U.S. Pat. No. 4,943,424; U.S. Pat. No. 5,087,347; U.S. Pat. No. 5,158,665; U.S. Pat. No. 5,208,005; U.S. Pat. No. 5,246,566; U.S. Pat. No. 5,716,897; and U.S. Pat. No. 5,851,949 are hereby incorporated by reference.

U.S. Pat. Nos. 5,444,032 and 5,608,968 teach a suitable bifunctional catalyst which is constituted by an amorphous silica-alumina gel and one or more metals belonging to Group VIIIA, and is effective in the hydroisomerization of long-chain normal paraffins containing more than 15 carbon atoms. An activated carbon catalyst support may also be used. U.S. Pat. Nos. 5,981,419 and 5,908,134 teach a suitable bifunctional catalyst which comprises: (a) a porous crystalline material isostructural with beta-zeolite selected from boro-silicate (BOR-B) and boro-alumino-silicate (Al-BOR-B) in which the molar SiO₂:Al₂O₃ ratio is higher than 300:1; (b) one or more metal(s) belonging to Group VIIIA, selected from platinum and palladium, in an amount comprised within the range of from 0.05 to 5% by weight. Article V. Calemma et al., App. Catal. A: Gen., 190 (2000), 207 teaches yet another suitable catalyst.

The isomerization and selective hydrocracking catalyst may be any of those well known in the art such as those described and cited above. Isomerization and selective cracking conditions include a temperature of about 150° C. to about 360° C. and a pressure of about 2 MPa absolute to about 4.7 MPa absolute. In another embodiment the isomerization conditions include a temperature of about 300° C. to about 360° C. and a pressure of about 3.1 MPa absolute to about 3.8 MPa absolute. Other operating conditions for the isomerization and selective hydrocracking zone are well known in the art. Some known isomerization catalysts, when operated under more severe conditions, also provide the selective hydrocracking catalytic function.

The isomerization and selective cracking zone effluent is processed through one or more separation steps to obtain two purified hydrocarbon streams, one useful as a diesel fuel or a diesel fuel blending component and the second useful as aviation fuel or an aviation fuel blending component. Depending upon the application, various additives may be combined with the diesel or aviation fuel composition generated in order to meet required specifications for different specific fuels. In particular, the aviation fuel composition generated herein complies with, is a blending component for, or may be combined with one or more additives to meet at least one of: ASTM D 1655 Specification for Aviation Turbine Fuels Defense Stan 91-91 Turbine Fuel, Aviation Kerosene Type, Jet A-1 NATO code F-35, F-34, F-37 Aviation Fuel Quality Requirements for Jointly Operated Systems (Joint Checklist) A combination of ASTM and Def Stan requirements GOST 10227 Jet Fuel Specifications (Russia) Canadian CAN/CGSB-3.22 Aviation Turbine Fuel, Wide Cut Type Canadian CAN/CGSB-3.23 Aviation Turbine Fuel, Kerosene Type MIL-DTL-83133, JP-8, MIL-DTL-5624, JP-4, JP-5 QAV-1 (Brazil) Especifcacao de Querosene de Aviacao No. 3 Jet Fuel (Chinese) according to GB6537 DCSEA 134A (France) Carbureacteur Pour Turbomachines D'Aviation, Type Kerosene Aviation Turbine Fuels of other countries, meeting the general grade requirements for Jet A, Jet A-1, Jet B, and TS-1 fuels as described in the IATA Guidance Material for Aviation Turbine Fuel Specifications. The aviation fuel is generally termed “jet fuel” herein and the term “jet fuel” is meant to encompass aviation fuel meeting the specifications above as well as to encompass aviation fuel used as a blending component of an aviation fuel meeting the specifications above. Additives may be added to the jet fuel in order to meet particular specifications. One particular type of jet fuel is JP-8, defined by Military Specification MIL-DTL-83133, which is a military grade type of highly refined kerosene based jet propellant specified by the United States Government. The fuel produced from glycerides or FAAs is very similar to isoparaffinic kerosene or iPK, also known as a synthetic jet fuel or synthetic paraffinic kerosene, SPK.

The specifications for different types of fuels are often expressed through acceptable ranges of chemical and physical requirements of the fuel. As stated above, aviation turbine fuels, a kerosene type fuel including JP-8, are specified by ML-DTL-83133, JP-4, a blend of gasoline, kerosene and light distillates, is specified by MIL-DTL-5624 and JP-5 a kerosene type fuel with low volatility and high flash point is also specified by MIL-DTL-5624, with the written specification of each being periodically revised. Often a distillation range from 10 percent recovered to a final boiling point is used as a key parameter defining different types of fuels. The distillations ranges are typically measured by ASTM Test Method D 86 or D2887. Therefore, blending of different components in order to meet the specification is quite common. While the product may meet fuel specifications, it is expected that some blending of the product with other blending components may be required to meet the desired set of fuel specifications. In other words, the aviation product is a composition which may be used with other components to form a fuel meeting at least one of the specifications for aviation fuel such as JP-8. The desired products are highly paraffinic distillate fuel components having a paraffin content of at least 75% by volume.

With the effluent stream of the isomerization and selective hydrocracking zone comprising a liquid component and a gaseous component, various portions of which may be recycled, multiple separation steps may be employed. For example, hydrogen may be first separated in an isomerization effluent separator with the separated hydrogen being removed in an overhead stream. Suitable operating conditions of the isomerization effluent separator include, for example, a temperature of 230° C. and a pressure of about 4.1 MPa absolute. If there is a low concentration of carbon oxides, or the carbon oxides are removed, the hydrogen may be recycled back to the hot high pressure hydrogen stripper for use both as a rectification gas and to combine with the remainder as a bottoms stream. The remainder is passed to the isomerization reaction zone and thus the hydrogen becomes a component of the isomerization reaction zone feed streams in order to provide the necessary hydrogen partial pressures for the reactor. The hydrogen is also a reactant in the deoxygenation reactors, and different feedstocks will consume different amounts of hydrogen. The isomerization effluent separator allows flexibility for the process to operate even when larger amounts of hydrogen are consumed in the first reaction zone. Furthermore, at least a portion of the remainder or bottoms stream of the isomerization effluent separator may be recycled to the isomerization reaction zone to increase the degree of isomerization.

The remainder of the isomerization effluent after the removal of hydrogen still has liquid and gaseous components and is cooled, by techniques such as air cooling or water cooling and passed to a cold separator where the liquid component is separated from the gaseous component. Suitable operating conditions of the cold separator include, for example, a temperature of about 20 to 60° C. and a pressure of about 3.8 MPa absolute. A water byproduct stream is also separated. At least a portion of the liquid component, after cooling and separating from the gaseous component, may be recycled back to the isomerization zone to increase the degree of isomerization. Prior to entering the cold separator, the remainder of the isomerization and selective hydrocracking zone effluent may be combined with the hot high pressure hydrogen stripper overhead stream, and the resulting combined stream may be introduced into the cold separator.

The liquid component contains the hydrocarbons useful as diesel fuel or diesel fuel blending components and aviation fuel or aviation fuel blending components, termed diesel boiling point range product and aviation boiling point range product, respectively, as well as smaller amounts of naphtha and LPG. The separated liquid component is further purified in a product distillation zone which separates lower boiling components and dissolved gases into an LPG and naphtha stream; an aviation range product; and a diesel range product. Suitable operating conditions of the product distillation zone include a temperature of from about 20 to about 200 C at the overhead and a pressure from about 0 to about 1.4 MPa absolute. The conditions of the distillation zone may be adjusted to control the relative amounts of hydrocarbon contained in the aviation range product stream and the diesel range product stream.

The LPG and naphtha stream may be further separated in a debutanizer or depropanizer in order to separate the LPG into an overhead stream, leaving the naphtha in a bottoms stream. Suitable operating conditions of this unit include a temperature of from about 20 to about 200° C. at the overhead and a pressure from about 0 to about 2.8 MPa absolute. The LPG may be sold as valuable product or may be used in other processes such as a feed to a hydrogen production facility. Similarly, the naphtha may be used in other processes, such as the feed to a hydrogen production facility.

The gaseous component separated in the product separator comprises mostly hydrogen and the carbon dioxide from the decarboxylation reaction. Other components such as carbon monoxide, propane, and hydrogen sulfide or other sulfur containing component may be present as well. It is desirable to recycle the hydrogen to the isomerization zone, but if the carbon dioxide was not removed, its concentration would quickly build up and effect the operation of the isomerization zone.

The carbon dioxide can be removed using the ionic liquid removal process as discussed above.

Similarly, a sulfur containing component such as hydrogen sulfide may be present to maintain the sulfided state of the deoxygenation catalyst or to control the relative amounts of the decarboxylation reaction and the hydrogenation reaction that are both occurring in the deoxygenation zone. The amount of sulfur is generally controlled and so must be removed before the hydrogen is recycled. The sulfur components may be removed using techniques such as absorption with an amine or by caustic wash.

The hydrogen remaining after the removal of at least carbon dioxide may be recycled to the reaction zone where hydrogenation primarily occurs and/or to any subsequent beds or reactors. The recycle stream may be introduced to the inlet of the reaction zone and/or to any subsequent beds or reactors. One benefit of the hydrocarbon recycle is to control the temperature rise across the individual beds. However, as discussed above, the amount of hydrocarbon recycle may be determined based upon the desired hydrogen solubility in the reaction zone which is in excess of that used for temperature control. Increasing the hydrogen solubility in the reaction mixture allows for successful operation at lower pressures, and thus reduced cost.

As discussed above, at least a portion of the diesel boiling point range product; at least a portion of the aviation boiling point range product, at least a portion of the LPG and naphtha stream; at least a portion of a naphtha stream or an LPG stream generated by separating the LPG and naphtha stream into an LPG stream and the naphtha stream; or any combination thereof may be recycled to the optional rectification zone of the hot high pressure hydrogen stripper.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for removing CO₂ from a gaseous mixture comprising CO₂, hydrocarbon vapor, and hydrogen comprising:
contacting the gaseous mixture with an absorbing medium in an absorption zone at a first pressure in range of about 2 MPa (g) to about 10 MPa (g) so that the CO₂ is absorbed by the absorbing medium forming a stream of absorbing medium rich in CO₂ and the hydrocarbon vapor, the stream of absorbing medium rich in CO₂ and the hydrocarbon vapor at a first pressure, the absorbing medium comprising an ionic liquid wherein the ionic liquid is not a pyrrolidinone-based ionic liquid;
reducing a pressure of the stream of absorbing medium rich in CO₂ and the hydrocarbon vapor from the first pressure to a second pressure lower than the first pressure to release the hydrocarbon vapor from the absorbing medium rich in CO₂ and the hydrocarbon vapor to form a stream of absorbing medium rich in CO₂;
reducing a pressure of the stream of absorbing medium rich in CO₂ from the second pressure to a third pressure lower than the second pressure to release the CO₂ from the absorbing medium to form a stream of absorbing medium substantially free of hydrocarbon vapor and CO₂.

2. The method of claim 1 further comprising cooling the stream of absorbing medium substantially free of hydrocarbon vapor and CO₂, compressing the stream of absorbing medium substantially free of hydrocarbon vapor and CO₂, and recycling the stream of cooled, compressed absorbing medium to the absorption zone.

3. The method of claim 1 further comprising recycling the hydrocarbon vapor to the absorption zone.

4. The method of claim 1 further comprising mixing the absorbing medium with the gaseous mixture in a mixing zone before the absorption zone.

5. The method of claim 1 further comprising recovering the CO₂, treating the CO₂, or both.

6. The method of claim 1 wherein the first pressure is in a range of about 2 MPa (g) to about 6 MPa (g), the second pressure is in a range of about 0.5 MPa (g) to about 2 MPa (g), and the third pressure is in a range of 0 MPa (g) to about 0.5 MPa (g).

7. The method of claim 1 wherein the gaseous mixture further comprises at least one of water, and CO, and further comprising recovering an overhead gas stream from the absorption zone.

8. The method of claim 1 wherein a cation of the ionic liquid comprises imidazolium, phosphonium, alcamine, guanidinium, tetraalkylammonium, pyrazolium, pyridinium, sulfonium, piperidinium, tetraalkylammonium, pyrazolium, pyridinium, sulfonium, or piperidinium.

9. The method of claim 1 wherein an anion of the ionic liquid comprises a carboxylate, an acetate, a tosylate, a cyanate, a halide, a sulfate, a hydrogen sulfate, a sulfonate, a sulfonyl imide, a phosphate, a borate, a carbonate, or a heterocyclic anion.

10. The method of claim 1 wherein the absorbing medium further comprises about 1 to about 90 wt % solvent.

11. The method of claim 1 further comprising regenerating the ionic liquid in the stream of adsorbing medium substantially free of hydrocarbon vapor and CO₂.

12. A method for processing a renewable feedstock comprising:
hydrotreating the renewable feedstock in a hydrotreating zone to produce a reactor effluent comprising hydrocarbons, hydrogen, water, CO and CO₂;
separating the reactor effluent in a separation zone at a first pressure in a range of about 2 MPa (g) to about 10 MPa (g) into a liquid stream comprising liquid hydrocarbon and a gaseous stream comprising hydrocarbon vapor, hydrogen, water, CO and CO₂;
contacting the gaseous mixture with an absorbing medium in an absorption zone so that the CO₂ is absorbed by the absorbing medium forming a stream of absorbing medium rich in CO₂ and the hydrocarbon vapor, the stream of absorbing medium rich in CO₂ and the hydrocarbon vapor at the first pressure, the absorbing medium comprising an ionic liquid wherein the ionic liquid is not a pyrrolidinone-based ionic liquid;
reducing a pressure of the stream of absorbing medium rich in CO₂ and the hydrocarbon vapor from the first pressure to a second pressure lower than the first pressure to release the hydrocarbon vapor from the absorbing medium rich in CO₂ and the hydrocarbon vapor to form a stream of absorbing medium rich in CO₂;
reducing a pressure of the stream of absorbing medium rich in CO₂ from the second pressure to a third pressure lower than the second pressure to release the CO₂ from the absorbing medium to form a stream of absorbing medium substantially free of hydrocarbon vapor and CO₂.

13. The method of claim 12 further comprising cooling the stream of absorbing medium substantially free of hydrocarbon vapor and CO₂, compressing the stream of absorbing medium substantially free of hydrocarbon vapor and CO₂, and recycling the stream of cooled, compressed absorbing medium to the absorption zone.

14. The method of claim 12 further comprising recycling the hydrocarbon vapor to the absorption zone.

15. The method of claim 12 further comprising mixing the absorbing medium with the gaseous mixture in a mixing zone before the absorption zone.

16. The method of claim 16 wherein the first pressure is in a range of about 2 MPa (g) to about 6 MPa (g), the second pressure is in a range of about 0.5 MPa (g) to about 2 MPa (g), and the third pressure is in a range of 0 MPa (g) to about 0.5 MPa (g).

17. The method of claim 12 wherein a cation of the ionic liquid comprises imidazolium, alcamine, guanidinium, tetraalkylammonium, pyrazolium, pyridinium, sulfonium, piperidinium, tetraalkylammonium, pyrazolium, pyridinium, sulfonium, or piperidinium.

18. The method of claim 12 wherein an anion of the ionic liquid comprises a carboxylate, an acetate, a tosylate, a cyanate, a halide, a sulfate, a hydrogen sulfate, a sulfonate, a sulfonyl imide, a phosphate, a borate, a carbonate, or a heterocyclic anion.

19. The method of claim 12 wherein the absorbing medium further comprises about 1 to about 90 wt % solvent.

20. The method of claim 12 further comprising regenerating the ionic liquid in the stream of adsorbing medium without hydrocarbon vapor or CO₂.
